(19) United States
(12) Patent Application Publication
(10) Pub. No.: US 2017/0296271 A1
(43) Pub. Date: Oct. 19, 2017

(54) MEDICAL SYSTEMS, DEVICES, AND RELATED METHODS

(71) Applicant: BOSTON SCIENTIFIC SCIMED, INC., MAPLE GROVE, MN (US)

(72) Inventor: Michael S.H. CHU, Brookline, MA (US)

(73) Assignee: BOSTON SCIENTIFIC SCIMED, INC., MAPLE GROVE, MN (US)

(21) Appl. No.: 15/485,885

(22) Filed: Apr. 12, 2017

Related U.S. Application Data
(60) Provisional application No. 62/322,615, filed on Apr. 14, 2016.

(51) Int. Cl. A61B 18/20; A61B 18/14; A61B 18/08; A61B 18/00

(52) U.S. Cl. CPC A61B 18/20; A61B 18/1477; A61B 18/1492; A61B 18/082; A61B 2018/00505; A61B 2018/1465; A61B 2018/0041

(57) ABSTRACT

A medical device may include a luminal elongate control member terminating in a first end effector. The medical device may further include a second end effector. The second end effector may be positioned within the luminal elongate control member. Further, the second end effector may be movable relative to the luminal elongate control member. Also, the medical device may be sheathless.

### CROSS-REFERENCE TO RELATED APPLICATIONS

This patent application claims the benefit of priority under 35 U.S.C. §119 to U.S. Provisional Patent Application No. 62/322,615, filed Apr. 14, 2016, which is herein incorporated by reference in its entirety.

### TECHNICAL FIELD

Various aspects of the present disclosure relate generally to medical systems, devices, and related methods. More specifically, the present disclosure relates to devices, systems, and methods for retrieving objects and/or performing therapies within a patient.

### BACKGROUND

Medical devices, such as baskets, are often used to remove organic material (e.g., blood clots, tissue, and biological concretions such as urinary, biliary, and pancreatic stones) and inorganic material (e.g., components of a medical device or other foreign matter), which may obstruct or otherwise be present within a patient's body cavities or passages. For example, concretions can develop in certain parts of the body, such as in the kidneys, pancreas, ureter, and gallbladder. Minimally invasive medical procedures are used to remove these concretions through natural orifices, or through an incision, such as during a percutaneous nephrolithotomy (“PNCL”) procedure. Medical devices are also used in lithotripsy and ureteroscopy procedures to treat urinary calculi (e.g., kidney stones) in the ureter of a patient.

Such medical devices may be passed through a working channel of a suitable insertion device (such as, e.g., an endoscope, ureteroscope, laparoscope, etc.) positioned in a body cavity in order to reach an operative site at a distal end of the insertion device. The size (e.g., radial dimension, diameter, and/or cross-sectional profile) of the medical device, such as, e.g., scissors, snares, forceps, baskets, laser fibers, and/or needles, therefore, may be limited by the diameter of the insertion device's working channel. Alternatively, the working channel of the insertion device may be increased so as to accommodate a medical device with a larger profile, which may inhibit both the flexibility of the insertion device and/or the ability of the insertion device to house other functional components (e.g., visualization devices, etc.), deliver one or more of irrigation or aspiration fluids, or may increase the outer diameter of the scope to an unusable size.

The systems, devices, and methods of the current disclosure may rectify some of the deficiencies described above or address other aspects of the prior art.

### SUMMARY

Examples of the present disclosure relate to, among other things, medical retrieval devices. Each of the examples disclosed herein may include one or more of the features described in connection with any of the other disclosed examples.

In one example, a medical device may include a luminal elongate control member terminating in a first end effector. The medical device may further include a second end effector. The second end effector may be positioned within the luminal elongate control member. Further, the second end effector may be movable relative to the luminal elongate control member. Also, the medical device may be sheathless.

Examples of the medical device may additionally and/or alternatively include one or more of the following features. A central longitudinal axis of the second end effector may be coaxial with a central longitudinal axis of the luminal elongate control member. Each of the first end effector and the second end effector may be independently actuatable. The medical device may further include a handle which may have a main body and a grip member, and the luminal elongate control member may be fixedly coupled to the main body. A proximal end of the second end effector may extend through an opening on a proximal end of the handle. A proximal end of the second end effector may be coupled to an energy connector, and wherein the second end effector may be configured to delivery energy to tissue of a patient. The first end effector may include a basket. The second end effector may include a laser fiber. The first end effector may include a snare. The second end effector may include a needle. At least a portion of the first end effector may include Nitinol. The first end effector may include a plurality of legs. The medical device may further include a connector having a lumen extending therethrough, wherein each of the luminal elongate control member and second end effector may extend through the lumen of the connector. The lumen of the connector may be tapered. The connector may be detachably coupled to a distal end of a handle.

In another example, a medical system may comprise an insertion device having a handle; a shaft coupled to the handle; and a port positioned on the handle an in fluid communication with a working channel of the shaft. The system may further include a sheathless medical device having a luminal elongate control member terminating in a first end effector. The medical device may also have a second end effector positioned within the luminal elongate control member. Additionally, the medical device may include a handle. Further, the system may include a connector having a first end coupled to the handle of the medical device and a second end coupled to the port of the insertion device.

Examples of the medical system may additionally and/or alternatively include one or more of the following features. Each of the first end effector and the second end effector may be independently moveable. The first end effector may include a basket. The second end effector may include a laser fiber. The first end effector may include a snare. The second end effector may include a needle. The connector may include a tapered lumen extending therethrough, wherein each of the luminal elongate control member and second end effector may extend through the tapered lumen of the connector.

In another example, a method for treating a patient may include delivering a shaft of an insertion device to a location within a patient. The shaft may be coupled to a handle of the insertion device and in fluid communication with a port on the handle of the insertion device. The method may further include positioning a sheathless medical device within the shaft of the insertion device, where the medical device may include a first end effector and a second end effector, where the second end effector may be moveable through a lumen of the first end effector. The method may also include moving the first end effector distally such that at least a portion of the first end effector is positioned distal of a distal end of the shaft. Further, the method may include moving the second end effector distally such that at least a portion of the second end effector is positioned distal of the distal end of the shaft. The first end effector and the second end effector may be independently movable relative to one another.

Examples of the method may additionally and/or alternatively include one or more of the following features. Moving the first end effector distally such that at least a portion of the second end effector is positioned distal of the distal end of the shaft may result in radial expansion of the first end effector. The method may further include energizing tissue at the location within the patient. The method may further include retracting the first end effector proximally to a position within the shaft of the insertion device and stabilizing a location of the second end effector with the first end effector. The first end effector may include one of a basket and a snare. The second end effector may include one of a laser fiber and a needle.

It may be understood that both the foregoing general description and the following detailed description are exemplary and explanatory only and are not restrictive of the features, as claimed.

As used herein, the terms “comprises,” “comprising,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” as used herein is used in the sense of “example,” rather than “ideal.”

### BRIEF DESCRIPTION OF THE DRAWINGS

The accompanying drawings, which are incorporated in and constitute a part of this specification, illustrate exemplary features of the present disclosure and together with the description, serve to explain the principles of the disclosure.

FIG. 1 illustrates an exemplary medical device;

FIG. 2 illustrates a cross-sectional view of a portion of the exemplary medical device of FIG. 1;

FIG. 3 illustrates a side-view of the medical device of FIGS. 1 and 2 coupled to an exemplary insertion device;

FIG. 4 illustrates an enlarged view of the medical device of FIGS. 1 and 2 coupled to the insertion device of FIG. 3, wherein the medical device is in an extended state;

FIG. 5 illustrates an enlarged view of the medical device of FIGS. 1 and 2 coupled to the insertion device of FIG. 3, wherein the medical device is in a retracted state;

FIG. 6 illustrates an exemplary connector according to aspects of the present disclosure;

FIG. 7 illustrates an exemplary end effector of a medical device according to aspects of the present disclosure;

FIG. 8 illustrates another exemplary end effector of a medical device according to aspects of the present disclosure;

FIGS. 9-13 illustrate various views of an exemplary multi-function medical device according to aspects of the present disclosure; and

FIGS. 14 and 15 illustrate various views of another exemplary multi-function medical device according to aspects of the present disclosure.

### DETAILED DESCRIPTION

Overview

Examples of the present disclosure relate to medical systems and devices for diagnosing and/or treating internal areas of a subject's body. The medical system may include an insertion device and one or more medical devices of reduced profile for introduction through the insertion device.

### DETAILED EXAMPLES

Reference will now be made in detail to examples of the present disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device or insertion device. When used herein, “proximal” refers to a position relatively closer to the exterior of the body or closer to a medical professional using the medical device or insertion device. In contrast, “distal” refers to a position relatively further away from the medical professional using the medical device or insertion device, or closer to the interior of the body.

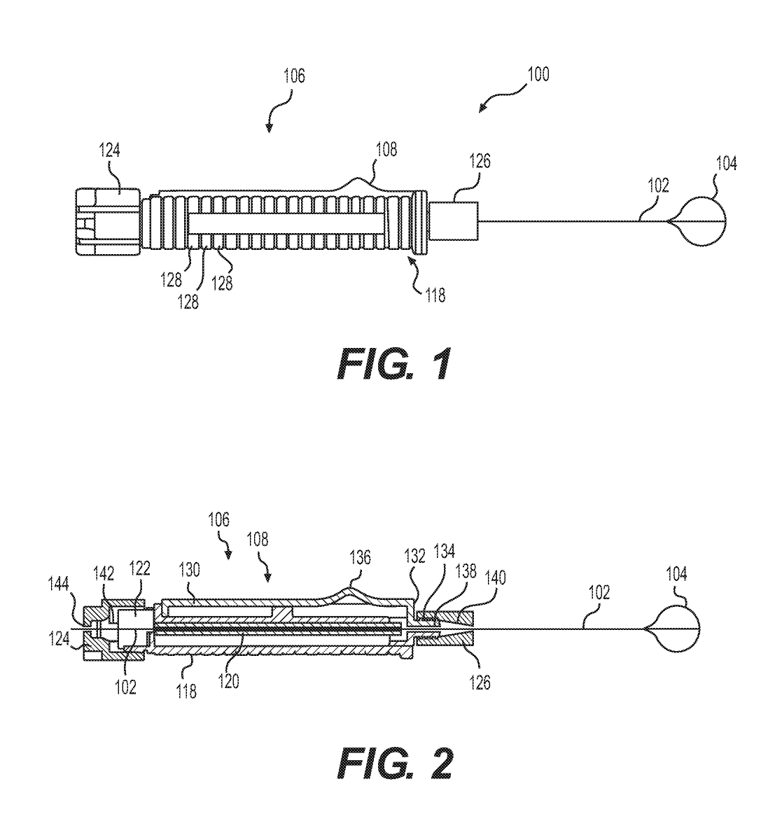

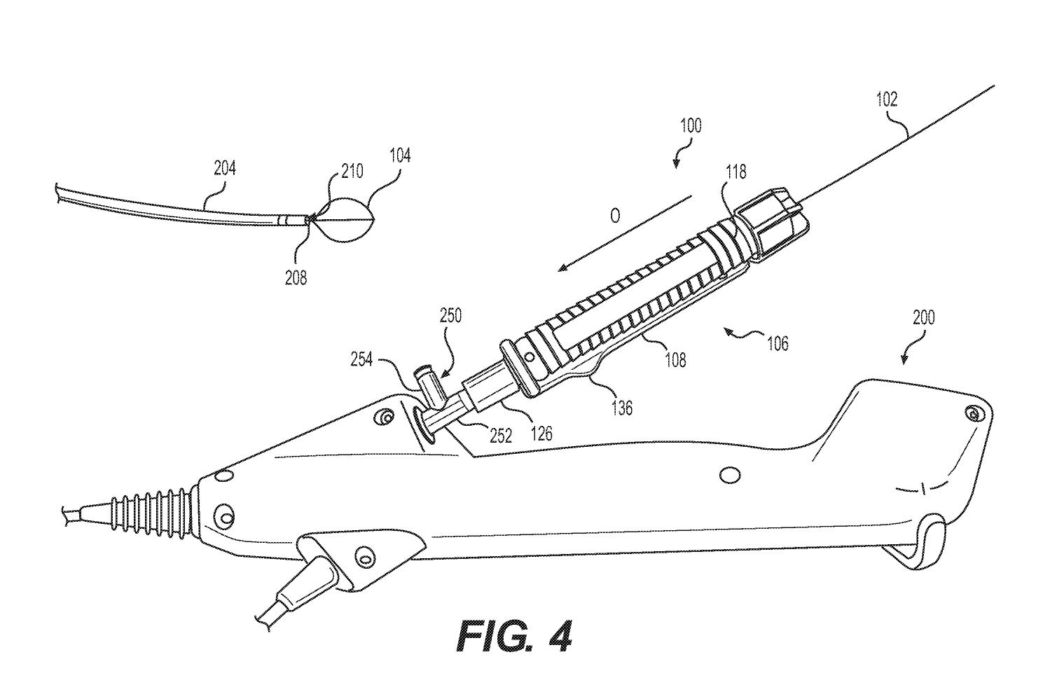

FIGS. 1 and 2 illustrate portions of an exemplary medical device 100. Medical device 100 may include an end effector 104 (e.g., a basket, snares, forceps, scissors, laser fibers, and/or needles) at the distal end of an elongate control member 102 (e.g., a wire, rod, hypotube, braid, shaft, and/or any other suitable drive member configured to transfer translational and/or rotational forces from its proximal end to its distal end). In some arrangements, end effector 104 may be movable between a radially collapsed configuration and a radially expanded configuration, as is described in further detail below. At least a portion of end effector 104 may be movable relative to a working channel 210 of insertion device 200 (FIG. 4) between an extended state and a retracted state, as will be described in further detail below. Medical device 100 may be a “sheathless” or “sheath-free” medical device in which no sheath, sleeve, or other such restraining member is positioned about (e.g., surrounding an exterior of medical device 100) such that expandable or otherwise actuatable portions of medical device 100 (e.g., end effector 104), if any, may freely expand toward an interior circumferential wall of working channel 210 within a shaft 204 of insertion device 200, while medical device is positioned in the retracted state. Accordingly, rather than movement of end effector 104 relative to a sheath to move end effector 104 between the extended (and optionally radially expanded) and retracted (and optionally radially compressed) states as is conventional, in the instant disclosure, end effector 104 of medical device moves between the extended (and optionally radially expanded) and retracted (and optionally radially compressed) states due to interaction between end effector 104 and working channel 210 of insertion device 200, as will be described in further detail below.

According to examples of the present disclosure, one or more portions of end effector 104 (or basket 304, forceps 324, basket 404, or snare 502, described below) may be made at least partially of a shape-memory material. Shape-memory material is a material that can be formed into a particular shape, retain that shape during resting conditions (e.g., when the shaped material is not subject to external forces or when external forces applied to the shaped material are insufficient to substantially deform the shape), be deformed into a second shape when subjected to a sufficiently strong external force, and revert substantially back to the initial shape when external forces are no longer applied. Examples of shape memory materials include synthetic plastics, stainless steel, and superelastic metallic alloys of nickel/titanium (e.g., Nitinol), copper, cobalt, vanadium, chromium, iron, or the like. Alternative end effector 104 materials include, but are not limited to, other metal alloys, powdered metals, ceramics, thermal plastic composites, ceramic composites, and polymers. Also, combinations of these and other materials can be used.

Medical device 100 may also include a handle assembly 106 at the proximal end of elongate control wire 102. Handle assembly 106 may include a grip member 108 moveable relative to main body 118 for moving end effector 104 between the extended state and the retracted state, as will be described in further detail below. Main body 118 may include one or more surface features or baffles 128 configured to aid a medical professional with securely grasping main body 118. Any number and arrangement of baffles 128 may be disposed on main body 118.

As shown in FIG. 2, main body 118 is configured to movably receive grip member 108 therein. Main body 118 may be hollow and have a semi-circular cross-sectional shape. Grip member 108 may be configured to be matingly received within main body 118. As shown in FIG. 2, grip member 108 may include a longitudinally extending member 130 and a distal facing end 132 including a nipple 134. Longitudinally extending member 130 may include a raised surface 136 to ease manipulation of the medical device 100 by the medical professional. For example, raised surface 136 may include a thumb or finger rest.

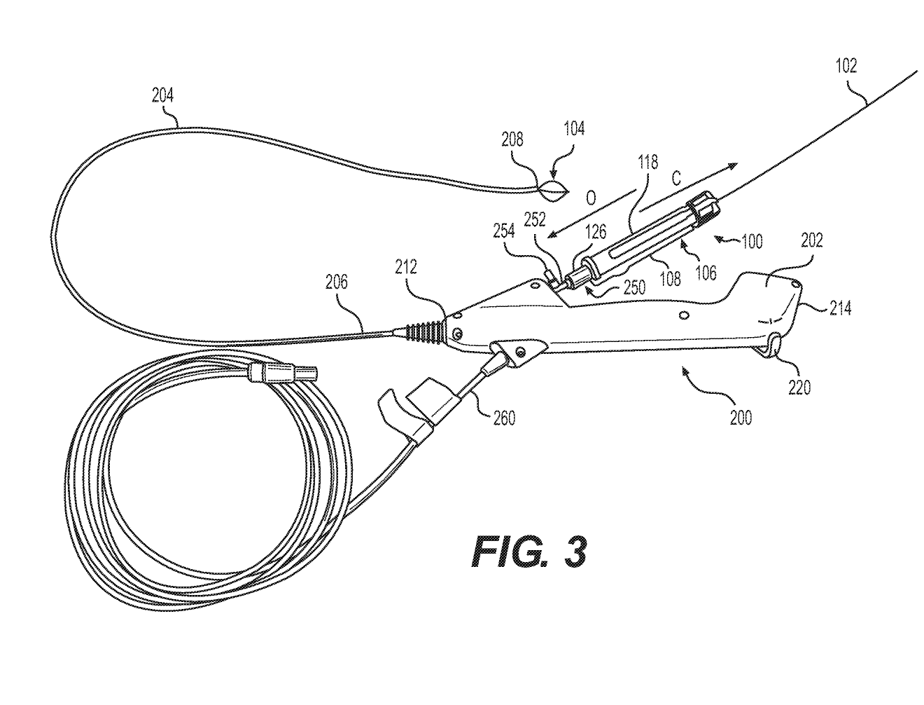

Nipple 134 may extend distally of distal facing end 132 and be configured to be coupled to a connector 126. For example, nipple 134 and connector 126 may be coupled via any appropriate means such as, for example, a screw fit connection or a friction fit connection. Accordingly, connector 126 and nipple 134 may be detachably connected. Connector 126 may include a male luer fitting. As shown in FIG. 2, elongate control member 102 may extend proximally through a lumen 140 in connector 126, through a lumen 138 in nipple 134, through a support tube 120 along longitudinally extending member 130, and towards a retention member 122. Retention member 122 may be coupled to main body 118 and may form a vise 142, or any other suitable holding mechanism. When vise 142 closes, elongate control member 102 may be fixedly coupled relative to main body 118. An end cap 124 may be placed onto proximal ends of holding member 122 and vise 142 to help close vise 142 around elongate control member 102. Vise 142 and/or retention member 122 may include an externally threaded portion (not shown), and end cap 124 may include complementary internal threading (not shown), such that end cap 124 may be screwed onto retention member 122 and vise 142. Elongate control member 102 may extend through an opening 144 in end cap 124 and proximally of handle assembly 106 as shown in FIGS. 3-5. Accordingly, if the medical professional determines a need to adjust a working length of elongate control member 102 relative to shaft 204, he/she may loosen (e.g., at least partially unscrew) end cap 124 from retention member 122 such that vise 142 may open sufficiently to enable elongate control member 102 to be extended or retracted relative to handle assembly 106.

FIG. 3 illustrates an exemplary insertion device 200 comprising a handle 202 coupled to shaft 204. Insertion device 200 may include any device configured to allow a medical professional to perform medical diagnoses and/or treatments on a subject. For example, insertion device 200 may include any device configured to allow a user to access and view internal areas of a subject's body. Additionally or alternatively, insertion device 200 may itself be a medical device and/or include any device configured to deliver medical devices 100, such as, for example, biopsy forceps, graspers, baskets, snares, probes, scissors, retrieval devices, lasers, and/or other tools, into a subject's body. Insertion device 200 may be inserted into one of a variety of body openings, lumens, and/or cavities. For example, insertion device 200 may be inserted into any portion of a urinary tract, such as a ureter, a gastrointestinal lumen, such as an esophagus, a vascular lumen, and/or an airway.

According to aspects of the present disclosure, insertion device 200 may be a ureteroscope. In some contemplated examples, insertion device 200 may be a sterile, single-use, and disposable ureteroscope. Alternatively, insertion device 200 may be a multiple-use, non-disposable ureteroscope. Other types of devices, however, may be substituted for the ureteroscope, including, as examples, an endoscope, a hysteroscope, a uteroscope, a bronchoscope, a cystoscope, and similar devices. Such devices may be single-use and disposable, or multiple-use and non-disposable.

Handle 202 of insertion device 200 may have any shape suitable for gripping and controlling insertion device 200. For example, handle 202 may have an ergonomic shape designed to be held comfortably in the hand, e.g., the palm of the hand. Shaft 204 may extend from a proximal end 206 to a distal end 208, such that proximal end 206 of shaft 204 may be coupled to (e.g., detachably or permanently connected to) a distal end 212 of handle 202. Handle 202 and/or shaft 204 may be disposable.

Insertion device 200 may include a steering mechanism for deflecting shaft 204 along one or more planes. For example, handle 202 may include an actuator 220 coupled to one or more portions of shaft 204 at or near distal end 208 of shaft 204 via one or more control members, such as steering wires (not shown). Any suitable steering mechanism and/or actuators may be used. Actuator 220 may be movably (e.g., pivotably) coupled to the handle 202 (e.g., via a rotatable post or shaft, not shown) such that rotating, pivoting, or moving actuator 220 moves distal end 208 of shaft 204 along a plane (e.g., left/right or up/down). According to some aspects, actuator 220 may include a locking device (not shown), such that actuator 220 may be locked in place when distal end 208 of shaft 204 is in a desired position.

Shaft 204 may include at least one working channel 210 (FIG. 4) extending between proximal end 206 of shaft 204 and distal end 208 of shaft 204. While only a single working channel 210 is depicted, e.g., in FIGS. 4 and 11-15, such a depiction is merely exemplary. For example, working channel 210 may be a primary working channel 210 for delivery of one or more medical devices 100, as will be described in further detail below. Shaft 204 may, however, include additional channels (not shown) extending between proximal end 206 and distal end 208 of shaft 204. Such additional channels may have different shapes and/or sizes. For example, working channel 210 may have a first cross-sectional size (e.g., diameter) or first shape (e.g., circular, ovular, etc.) while one or more additional channels may have a second cross-sectional size (e.g., diameter) or second shape (e.g., circular, ovular, etc.) different than the first size and first shape. In some examples, shaft 204 may include one or more electronic components, such as a camera or other imaging device, a light source, and/or other sensor (not shown). Additionally or alternatively, one of the additional channels may provide a lumen for light delivery and/or steering control members (not shown) associated with actuator 220 for steering shaft 204.

Handle 202 may include at least one port coupling 250 (e.g., a T-shaped or Y-shaped luer port connection). Port coupling 250 may include a first branch 252 in communication with working channel 210, e.g., to allow for the insertion of one or more medical device(s) 100 through working channel 210 to distal end 208 of shaft 204. For example, first branch 252, as shown in FIG. 3, may be coupled to connector 126 through which end effector 104 may be delivered into first branch 252 and working channel 210. Connector 126 may be coupled to first branch 252 through any appropriate means (e.g., screw fit, friction fit, etc.). Port coupling 250 may also include a second branch 254. Second branch 254 may be fluidly coupled to one or more sources of irrigation and/or suction fluid. Accordingly, irrigation fluid (not shown) may be delivered (e.g., pumped) through working channel 210, via second branch 254 to provide lubrication and/or aid in visualization. Handle 202 may also include an umbilicus hub or connector 260 for facilitating electrical connections and functions, such as transferring data and/or powering a light source.

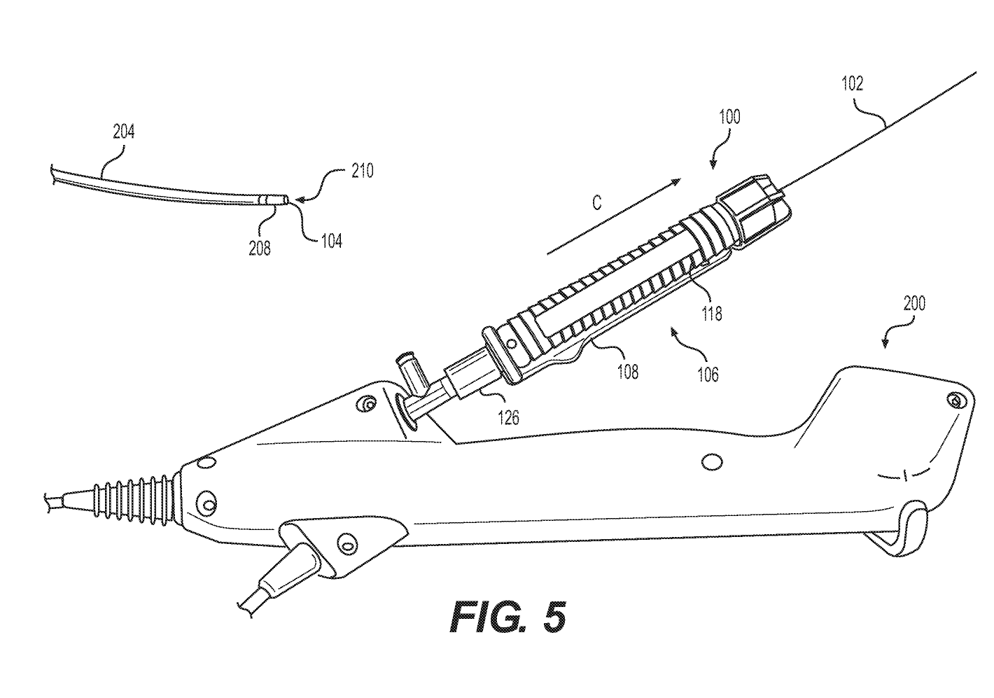

As shown in FIG. 3, and as described above, main body 118 of handle assembly 106 is moveable relative to grip member 108 of handle assembly 106 in a first direction O and a second direction C. That is, as shown in FIG. 4, when main body 118 is urged in first direction O relative to grip member 108 of handle assembly 106, end effector 104 is moved toward the extended state, in which at least end effector 104 extends through working channel 210 distally of distal end 208 of shaft 204. In contrast, as shown in FIG. 5, when main body 118 is urged in second direction C relative to grip member 108 of handle assembly 106, end effector 104 is moved toward the retracted state, in which at least end effector 104 is positioned within working channel 210 proximally of distal end 208 of shaft 204.

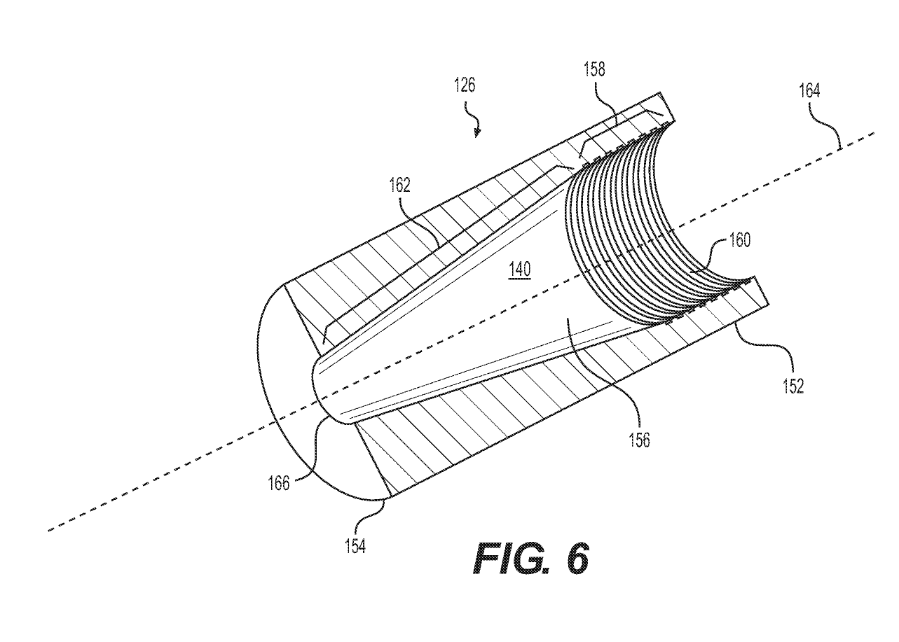

As noted above, connector 126 may include a male luer fitting. As shown in FIG. 6, for example, lumen 140 of connector 126 may be at least partially defined by an inner circumferential wall 156 extending between a proximal end 152 and a distal end 154 of connector 126. A first portion 158 of inner circumferential wall 156 adjacent proximal end 152 may include a screw thread 160 for selectively coupling and uncoupling connector 126 to/from nipple 134 of handle assembly 106. A second portion 162 of inner circumferential wall extending distally of first portion 158 may be narrowed, or angled relative to central longitudinal axis 164 of connector 126. That is, lumen 140 may be tapered distally such that a cross-sectional size (e.g., diameter) of a distal-most end 166 of lumen 140 is smaller than a cross-sectional size (e.g., diameter) of lumen 140 proximal of distal-most end 166. Accordingly, upon the insertion of end effector 104 and/or elongate control member 102 through connector 126 for delivery through working channel 210, tapered lumen 140 of connector 126 may facilitate guiding or directing end effector 104 and/or elongate control member 102 through distal-most end 166 of connector 126 and into working channel 210. In the case of a basket or snare, tapered lumen 140 of connector 126 may facilitate collapsing the basket or snare end effector 104 into working channel 210.

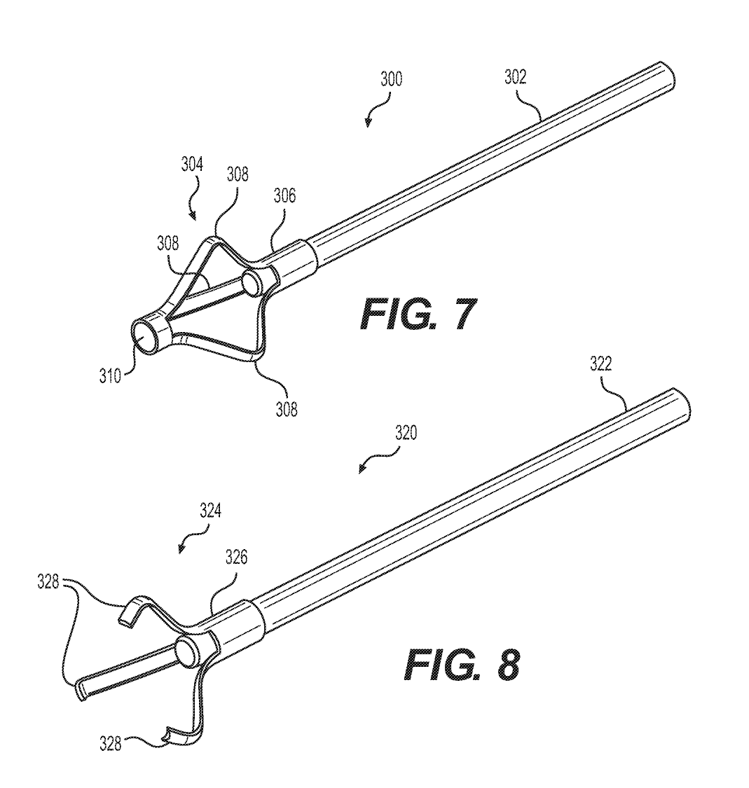

FIG. 7 illustrates a first exemplary medical device 300 according to aspects of this disclosure. As shown in FIG. 7, medical device 300 may include an elongate control member 302, similar in construction and function to elongate control member 102, described above. In addition, medical device 300 may include an end effector comprising a basket 304, coupled to elongate control member 302 in any appropriate manner (e.g., welding, adhesives, friction fit, mechanical fasteners, and the like). For example, a method of basket 304 formation may involve starting with a hollow tube or cannula, such as tubular member 306. At least a portion of tubular member 306 may then be cut (e.g., laser cut) with a cut pattern to produce one or more legs 308. While three legs 308 are shown in FIG. 7, it is understood that a greater or lesser number of legs 308 may be formed from tubular member 306. For example, a construction with two legs 308 may form a snare device (not shown). For axial basket 304 features (e.g., features extending substantially axially along a longitudinal axis of the underlying basket 304 structure) the cut pattern may include slots cut lengthwise along the exterior of a wall of tubular member 306 substantially parallel to a longitudinal axis of tubular member 306. For helical, spiral, or other features that are not solely axial in direction (e.g., features that extend at an angle with respect to a longitudinal axis of tubular member 306), the cut pattern may include slots cut with both an axial component and a radial component. After cutting, any one or more portions of the resultant legs 308 may be further manufactured and/or machined.

Following cutting and optional further machining and/or cleaning, the resulting form may be compressed lengthwise (e.g., expanded radially) to spread legs 308 and manipulate legs 308 into a desired shape. This shape may then be heat treated (e.g., annealed) into the material to form a desired basket 304 configuration. Accordingly, basket 304 may be monolithically formed single piece of material. While the foregoing description details an exemplary method of manufacture of basket 304, other suitable methods may be used without departing from the scope of this disclosure. As shown in FIG. 7, medical device 300 is sheathless or sheath-free. Accordingly, radial expansion and collapse of basket 304 may be effected by extension and retraction of basket 304 relative to working channel 210 of shaft 204. For example, extension of basket 304 distally of distal end 208 of shaft 204 causes basket 304 to move toward the extended state in which legs 308 of basket 304 may expand radially outward towards the configuration depicted in FIG. 7. Additionally, retraction of basket 304 into distal end 208 of shaft 204 may cause basket 304 to contract or collapse and move towards the retracted stated, as shown in FIG. 5.

FIG. 8 illustrates another exemplary medical device 320 according to aspects of this disclosure. As shown in FIG. 8, medical device 320 may include an elongate control member 322, similar in construction and function to elongate control members 102 and 302, described above. In addition, medical device 320 may include an end effector comprising a forceps 324 coupled to elongate control member 322 in any appropriate manner (e.g., welding, adhesives, friction fit, mechanical fasteners, and the like). Forceps 324 may be formed in a similar manner as described above for basket 304. Accordingly, a tubular member 306 may be cut (e.g., laser cut) to form two or more arms 328. For example, as shown in FIG. 8, tubular member 306 may be cut to form three arms 328. While three arms 328 are shown in FIG. 8, it is understood that a greater or lesser number of arm 328 may be formed from tubular member 326. Similar to medical devices 100 and 300 described above, medical device 320 is sheathless or sheath-free medical device. Accordingly, radial expansion and collapse of forceps 324 may be effected by extension and retraction of forceps 324 relative to working channel 210 of shaft 204. For example, extension of forceps 324 distally of distal end 208 of shaft 204 causes forceps 324 to move toward the extended state in which arms 328 of forceps 324 may expand radially outward towards the configuration depicted in FIG. 8. Additionally, retraction of forceps 324 into distal end 208 of shaft 204 may cause forceps 324 to contract or collapse and move towards the retracted stated, as shown in FIG. 5.

As noted above, medical devices 100, 300, and 320 are sheathless or sheath-free medical devices in that no sheath or other such radially constraining member surrounds medical devices 100, 300, and 320 (including end effector 104, basket 304, and forceps 324) other than shaft 204 when coupled to insertion device 200. Accordingly, a cross-sectional size, dimension, or profile of medical device 100 is reduced relative to conventional medical devices. As such, an amount of available space (or real estate) within working channel 210 that is unoccupied by medical device 100 may be increased relative to conventional medical devices. This relatively increased available real estate within working channel 210 of shaft 204 may facilitate an increased flow of irrigation or other such fluid (e.g., via second branch 254) through working channel 210 of shaft 204 and/or evacuation of material (e.g., organic or inorganic material located within the body of the patient). Additionally or alternatively, the relatively increased available real estate within working channel 210 of shaft 204 may facilitate insertion of a second (or more) medical devices alongside medical device 100, 300, and 320.

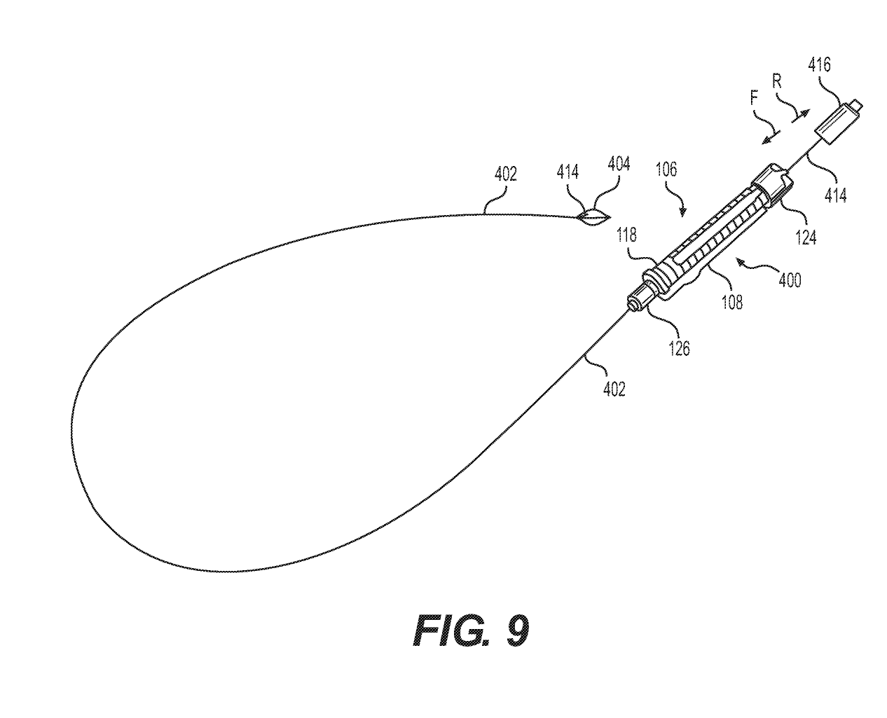

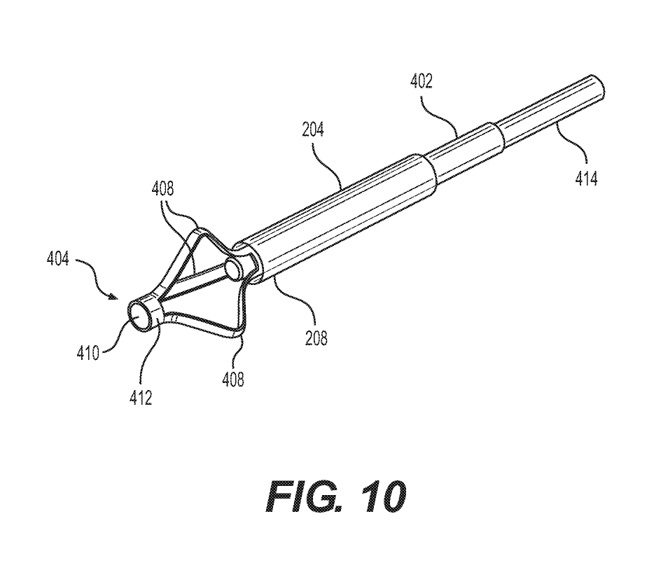

To further reduce the necessary real estate for delivery of medical devices, one or more medical devices may be combined to perform multiple therapies without the necessity of separate delivery mechanisms. For example, FIGS. 9-13 depict an exemplary multi-function or combination medical device 400 according to aspects of the present disclosure. Medical device 400 may be, for example, a combination basket 404 and laser fiber 406. In such an arrangement, medical device 400 may include a luminal (e.g., tubular, hollow, etc.) elongate control member 402 extending from handle assembly 106 and defining a lumen 410 (FIG. 10). Luminal elongate control member 402 may be fixedly secured to one or more of main body 118 and/or retention member 122 of handle assembly 106 in any appropriate manner (e.g., adhesives, welding, crimping, and/or mechanical fasteners, etc.). In some arrangements, luminal elongate control member 402 may be fixedly secured to nipple 134. Accordingly, extension and retraction of luminal elongate control member 402 may be controlled via movement of main body 118 in directions O and C, described above.

As shown in FIG. 10, luminal elongate control member 402 may include an end effector 404 on a distal end thereof. For example, end effector 404 may be a basket 404, similar in construction to basket 304 described above. Accordingly, a plurality of legs 408 may be formed (e.g., laser cut) through a distal end portion of luminal elongate control member 402 in a manner similar to the formation of legs 308 along a portion of tubular member 306, described above. Formation of legs 408 through luminal elongate control member 402 may form a distal collar 412 through which lumen 410 of luminal elongate control member 402 may extend, as will be described in further detail below.

In addition to basket 404, combination medical device 400 may include an energy transmission element 414 extending through lumen 410 of luminal elongate control member 402. Energy transmission element 414 may include any appropriate mechanism for delivery of energy therethrough for performing one or more therapies (e.g., lithotripsy). For example, energy transmission element 414 may be configured for delivery of one or more of thermal, chemical, electrical, electromagnetic (e.g., light), sound, nuclear, kinetic, or potential energy to treat a patient. By way of example only, energy transmission element 414 may comprise a laser fiber 414.

Laser fiber 414 may be movably positioned within luminal elongate control element 402. That is, laser fiber 414 may extend through opening 144 (FIG. 2) in end cap 124, proximally of handle assembly 106, and be operably coupled to an energy connector 416 as shown in FIG. 9. Energy connector 416 may be any available laser connector 416 such as, for example, a laser fiber connector for coupling laser fiber 414 to a source of laser energy, without departing from the scope of this disclosure. Additionally, energy connector 416 may include a luer fitting, hemostasis valve, or other valve (not shown) configured to seal around laser fiber 414 and releasably hold laser fiber 414 in place, and may include a side port (e.g., for aspiration). If the medical professional determines a need to adjust a working length of laser fiber 414 relative to shaft 204 and/or a position of laser fiber 414 relative to basket 404, he/she may loosen (e.g., at least partially unscrew) end cap 124 from retention member 122 such that vise 142 may open sufficiently to enable laser fiber 414 to be extended or retracted relative to handle assembly 106 (e.g., in direction F or R) so as to axial advance and/or retract laser fiber 414 to perform one or more therapies. That is, upon loosening vise 142, laser fiber 414 may be advanced and retracted as shown in FIG. 9. Additionally, as luminal elongate control member 402 is fixedly secured to one or more of main body 118 and/or retention member 122, luminal elongate control member 402 may remain stationary while laser fiber 414 is extended and retracted relative to handle assembly 106. By way of example only, laser fiber 414 may be advanced or retracted relative to basket 404 to facilitate a lithotripsy procedure to fragment or otherwise disrupt material (e.g., stones or other concretions) within the body of a patient. For example, in some arrangements, a medical professional may manipulate basket 404 so as to capture or retain material to be disrupted while he/she activates laser fiber 414 to disrupt, fragment, or dust the material captured within basket 404.

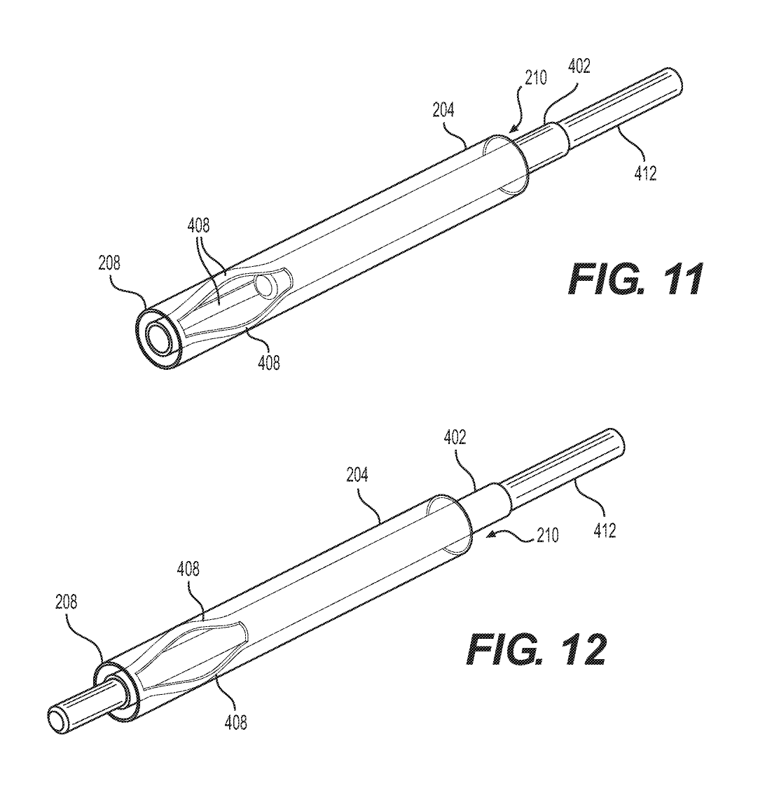

Additionally, as depicted in FIGS. 11 and 12, each of basket 404 and laser fiber 414 are independently actuatable. Accordingly, laser fiber 414 may be used alone, and/or deployed prior to movement of basket 404 toward the extended state. In such an arrangement, basket 404 may be used to stabilize or center laser fiber 414 relative to shaft 204. For example, main body 118 of handle assembly basket 404 may be retracted or maintained in the retracted configuration (as shown in FIG. 5). In such a configuration, legs 408 of basket 404 may expand radially outwardly (e.g., bow outwardly) towards an inner circumferential wall of shaft 204 as shown in FIG. 11. Accordingly, interaction of legs 408 of basket 404 with the inner circumferential wall of shaft 204 may stabilize, secure, or otherwise maintain basket 404 in place. While basket 404 is maintained in position within working channel 210 of shaft 204, laser fiber 414 may be advanced through lumen 410 of distal collar 412 and distally of distal end 208 of shaft 204 as shown in FIG. 12. In such an arrangement, laser fiber 414 may be stabilized relative to shaft 204 for increasingly accurate energy delivery. It is understood that any material disrupted by laser fiber 414 during delivery of energy thereto (e.g., stone fragments or stone dust) may be retracted through a channel defined between an inner circumferential wall of shaft 204 and an exterior (e.g., radially outermost) surface of luminal elongate control member 402. That is, a channel formed by the radial space between luminal elongate control member 402 and shaft 204 may be coupled to a source of negative pressure so as to form a return channel for extraction of such disrupted material.

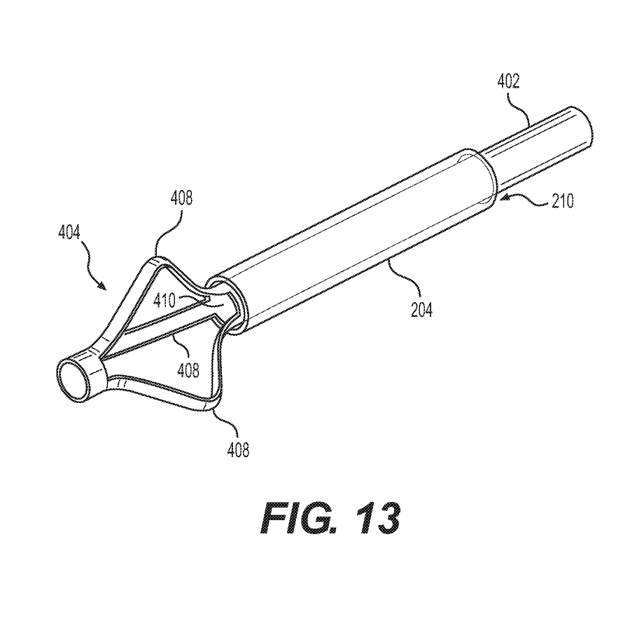

Optionally, following extension of laser fiber 414 through distal collar 412, basket 404 may additionally be extended distally of distal end 208 of shaft 204 so as to allow both of basket 404 and laser fiber 414 to be used in conjunction with one another. Additionally or alternatively, as shown in FIG. 13, laser fiber 414 may be removed from lumen 410 of luminal elongate control member 402. In such an arrangement, shaft 204 and luminal elongate control member 402 may form an irrigation circuit for delivery and removal of irrigation fluid. In such a manner, irrigation fluid may be delivered through working channel 210 of shaft 204 between an inner circumferential wall of shaft 204 and an exterior (e.g., radially outermost) surface of luminal elongate control member 402. That is, a channel formed by the radial space between luminal elongate control member 402 and shaft 204 may comprise an irrigation fluid delivery channel. Additionally, lumen 410 of luminal elongate control member 402 may be coupled to a source of negative pressure so as to form an irrigation return channel. Since the size (e.g., diameter) of lumen 410 of luminal elongate control member 402 remains constant throughout use, a medical professional may maintain and stabilize the pressure and fluid volume of irrigation fluid within the patient during a procedure.

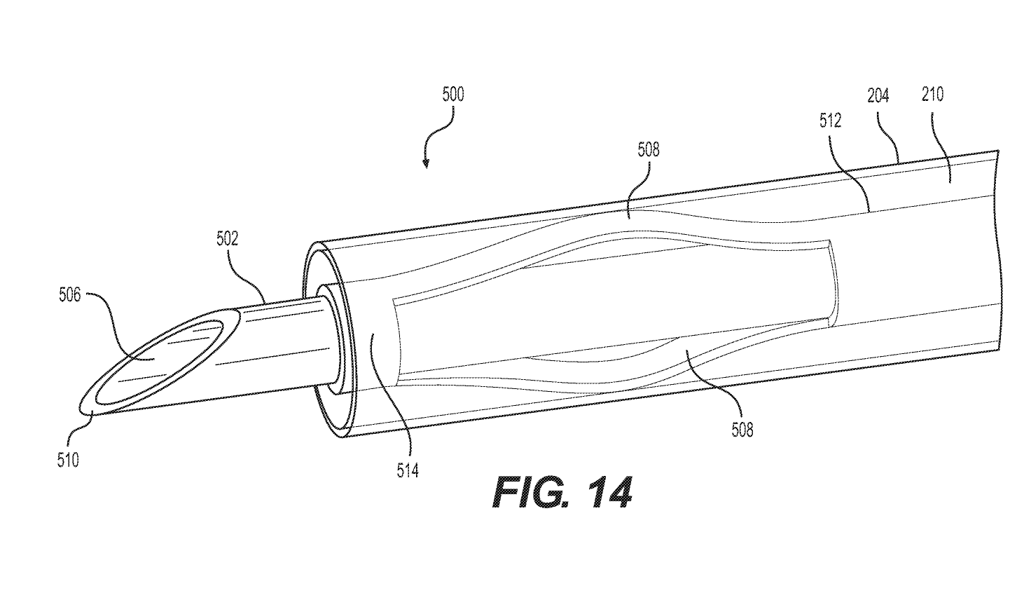

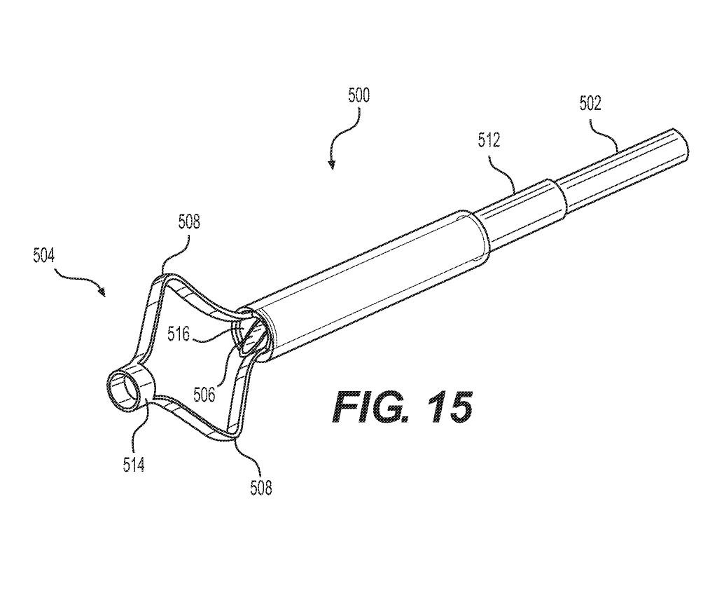

FIGS. 14 and 15 illustrate a further exemplary multi-function or combination medical device 500 according to further aspects of the present disclosure. Medical device 500 may be, for example, a combination snare 504 and needle 502. In such an arrangement, needle 502 may define a needle lumen 506 for delivery of one or more fluids (e.g., saline, medicament, hydrogel, etc.) and may terminate in a sharp tip 510 configured to facilitate tissue piercing. A proximal end of needle 502 may extend through a proximal end of handle assembly 106 and through end cap 124, in a manner similar to that as described above for laser fiber 414 above. That is, needle 502 may extend through opening 144 in end cap 124. If the medical professional determines a need to adjust a working length of needle 502 relative to shaft 204 and/or a position of needle 502 relative to snare 504, he/she may loosen (e.g., at least partially unscrew) end cap 124 from retention member 122 such that vise 142 may open sufficiently to enable needle 502 to be extended or retracted relative to handle assembly 106 so as to axial advance and/or retract needle 502 to perform one or more therapies.

That is, needle 502 may be movably positioned within a luminal elongate control element 512 of snare 504. Luminal elongate control member 512 may be fixedly secured to one or more of main body 118 and/or retention member 122 of handle assembly 106 in any appropriate manner (e.g., adhesives, welding, crimping, and/or mechanical fasteners, etc.). Accordingly, extension and retraction of luminal elongate control member 512 may be controlled in a manner similar to that described above for elongate control member 102 of medical device 100, as will be described in further detail below.

As shown in FIGS. 14 and 15, luminal elongate control member 512 may include snare 504 on a distal end thereof. For example, snare 504 may be similar in construction to basket 304 described above. Accordingly, a plurality of legs 508 may be formed (e.g., laser cut) through a distal end portion of luminal elongate control member 512 in a manner similar to the formation of legs 308 along a portion of tubular member 306, described above. Formation of legs 508 through luminal elongate control member 512 may form a distal collar 514 through which lumen 516 of luminal elongate control member 512 may extend, as will be described in further detail below.

Additionally, as depicted in FIGS. 14 and 15, each of snare 504 and needle 502 are independently actuatable. Accordingly, needle 502 may be used alone, and/or deployed prior to movement of snare 504 toward the extended state. In such an arrangement, snare 504 may be used to stabilize or center needle 502 relative to shaft 204. For example, main body 118 of handle assembly basket 404 may be retracted or maintained in the retracted configuration (as shown in FIG. 14). In such a configuration, legs 508 of snare 504 may expand radially outwardly (e.g., bow outwardly) towards an inner circumferential wall of shaft 204 as shown. Accordingly, interaction of legs 508 with the inner circumferential wall of shaft 204 may stabilize, secure, or otherwise maintain snare 504 in place. While snare 504 is maintained in position within working channel 210 of shaft 204, needle 502 may be advanced through lumen 516 and through distal collar 514 and distally of distal end 208 of shaft 204 as shown in FIG. 14. In such an arrangement, needle 502 may be stabilized relative to shaft 204 for increasingly accurate delivery of material through needle 502. Optionally, following extension of needle 502 through distal collar 514, snare 504 may additionally be extended distally of distal end 208 of shaft 204 so as to allow both of snare 504 and needle 502 to be used in conjunction with one another. Additionally or alternatively, needle 502 may be removed from lumen 516 of luminal elongate control member 512. Further, it is understood that a proximal end of needle 502 may be coupled to an energy connector for delivery of energy through needle 502 for cauterization (or other therapies) of tissue.

While FIGS. 9-15 illustrate various sheathless medical devices, it is understood that such examples are for explanation purposes only. Indeed, while FIGS. 9-13 illustrate combination medical device 400 including a laser fiber and a basket and FIGS. 14 and 15 illustrate combination medical device 500 including a snare and a needle, such arrangements are merely examples. Rather, other such combination medical device may include a basket and a needle, a snare and a laser fiber, a forceps and a laser fiber, a forceps and a needle, a needle and a laser fiber, etc.

In use, a medical professional may insert an insertion device 200 to a location within the subject's body. If the medical professional determines a need to perform one or more therapies, he/she may deliver a medical device through a working channel 210 of a shaft 204 of insertion device 200 for performing such therapies. Such medical devices may include any one or more of medical devices 100, 300, 320, 400, or 500 described above. Due to the arrangement of each of such medical devices being sheathless or sheath-free, and optionally, a combination medical device, a profile (e.g., size, cross-sectional dimension, diameter, etc.) of such a medical device may be reduced relative to conventional medical devices. In such a manner, available space or real estate within working channel 210 of shaft 204 may be employed for other uses. For example, available space or real estate within working channel 210 of shaft 204 may be used for delivery or extraction of irrigation fluids, aspiration fluids, and/or disrupted material (e.g., stone fragments or stone dust). Additionally or alternatively, available space or real estate within working channel 210 of shaft 204 may be used for delivery of additional medical devices.

While principles of the present disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

We claim:

1. A medical device, comprising:
a luminal elongate control member terminating in a first end effector; and
a second end effector positioned within the luminal elongate control member and movable relative thereto,
wherein the medical device is sheathless.

2. The medical device of claim 1, wherein each of the first end effector and the second end effector are independently actuatable.

3. The medical device of claim 1, further including a handle, having:
a main body; and
a grip member,
wherein the luminal elongate control member is fixedly coupled to the main body.

4. The medical device of claim 1, wherein first end effector includes a basket.

5. The medical device of claim 4, wherein the second end effector includes a laser fiber.

6. The medical device of claim 1, wherein the first end effector includes a snare.

7. The medical device of claim 6, wherein the second end effector includes a needle.

8. A medical system, comprising:
an insertion device having:
a handle;
a shaft coupled to the handle; and
a port positioned on the handle and in fluid communication with a working channel of the shaft;
a sheathless medical device having:
a luminal elongate control member terminating in a first end effector;
a second end effector positioned within the luminal elongate control member; and
a handle; and
a connector having a first end coupled to the handle of the medical device and a second end coupled to the port of the insertion device.

9. The medical system of claim 8, wherein the first end effector and the second end effector are independently moveable relative to one other.

10. The medical system of claim 8, wherein the first end effector includes a basket.

11. The medical system of claim 10, wherein the second end effector includes a laser fiber moveable through the basket.

12. The medical system of claim 8, wherein the first end effector includes a snare.

13. The medical system of claim 12, wherein the second end effector includes a needle moveable through the snare.

14. The medical system of claim 8, wherein the connector includes a tapered lumen extending therethrough, and wherein each of the luminal elongate control member and second end effector extend through the tapered lumen of the connector.

15. A method for treating a patient, comprising:
delivering a shaft of an insertion device to a location within a patient, the shaft being coupled to a handle of the insertion device and in fluid communication with a port on the handle of the insertion device;
positioning a sheathless medical device within the shaft of the insertion device, wherein the medical device includes a first end effector and a second end effector, and wherein the second end effector is moveable through a lumen of the first end effector; and
moving the first end effector distally such that at least a portion of the first end effector is positioned distal of a distal end of the shaft; and
moving the second end effector distally such that at least a portion of the second end effector is positioned distal of the distal end of the shaft,
wherein the first end effector and the second end effector are independently movable relative to one another.

16. The method of claim 15, wherein moving the first end effector distally such that at least a portion of the first end effector is positioned distal of the distal end of the shaft results in radial expansion of the first end effector.

17. The method of claim 15, further including:
energizing tissue at the location within the patient.

18. The method of claim 15, further including:
retracting the first end effector proximally to a position within the shaft of the insertion device; and
stabilizing a location of the second end effector with the first end effector.

19. The method of claim 15, wherein the first end effector includes one of a basket and a snare.

20. The method of claim 15, wherein the second end effector includes one of a laser fiber and a needle.
